(19) United States
(12) Patent Application Publication
(10) Pub. No.: US 2011/0190726 A1
(43) Pub. Date: Aug. 4, 2011

(54) Method and apparatus for micro-needle array electrode treatment of tissue

(75) Inventors: Basil M. Hantash, East Palo Alto, CA (US); Joseph L. Dallarosa, Redwood City, CA (US); D. Bommi Bommannan, Los Altos, CA (US)

(73) Assignee: RELIANT TECHNOLOGIES, INC., Hayward, CA (US)

(21) Appl. No.: 13/085,971

(22) Filed: Apr. 13, 2011

Related U.S. Application Data
(62) Division of application No. 11/564,250, filed on Nov. 28, 2006.
(60) Provisional application No. 60/741,031, filed on Nov. 29, 2005.

(51) Int. Cl. A61M 37/00; A61N 5/00

(52) U.S. Cl. 604/506; 607/100

(57) ABSTRACT

Systems and methods for revitalizing aging skin using electromagnetic energy that is delivered using a plurality of needles that are capable of penetrating the skin to desired depths. A particular aspect is the capability to spare zones of tissue from thermal exposure. This sparing of tissue allows new tissue to be regenerated while the heat treatment can shrink the collagen and tighten the underlying structures. Additionally, the system is capable of delivering therapeutically beneficial substances either through the penetrating needles or through channels that have been created by the penetration of the needles.

### CROSS-REFERENCE TO RELATED APPLICATIONS

This application is a divisional of application Ser. No. 11/564,250, filed Nov. 28, 2006, which claims the benefit of application Ser. No. 60/741,031, filed Nov. 29, 2005. Each of these applications is hereby incorporated by reference herein in its entirety for all purposes.

### BACKGROUND OF THE INVENTION

This invention relates generally to biological tissue treatment using electromagnetic energy delivered through an array of needle electrodes. More particularly, it relates to using radio frequency energy through an array of microneedles for rejuvenating human skin by a fractional treatment.

Skin is the primary barrier that withstands environmental impact, such as sun, cold, wind, etc. Along with aging, environmental factors cause the skin to lose its youthful look and develop wrinkles Human skin is made of epidermis, which is about 100 μm thick, followed by the dermis, which can extend up to 4 mm from the surface and finally the subcutaneous layer. These three layers control the overall appearance of the skin (youthful or aged). The dermis is made up of elastin, collagen, glycosoaminoglycans, and proteoglycans. The subcutaneous layer also has fibrous vertical bands that course through it and represent a link between dermal collagen and the subcutaneous layer. The collagen fibers provide the strength and elasticity to skin. With age and sun exposure, collagen loses its elasticity (tensile strength) and skin loses its youthful, tight appearance. Not surprisingly, numerous techniques have been described for rejuvenating the appearance of skin.

One approach to skin rejuvenation is to physically inject collagen into the skin. This gives an appearance of fullness or plumpness and the offending lines are smoothened. Bovine collagen has been used for this purpose. Unfortunately, this is not a long-lasting or complete fix for the problem and there are frequent reports of allergic reactions to the collagen injections.

It is now well established that collagen is sensitive to heat treatment and denatures when heated above its transition temperature. This denaturing is accompanied by shrinking of the collagen fibers and this shrinking can provide sagging or wrinkled skin with a tightened youthful appearance. Both heat and chemical based approaches have been described and used to shrink collagen.

Peeling most or all of the outer layer of the skin is another known method of rejuvenating the skin Peeling can be achieved chemically, mechanically or photothermally. Chemical peeling is carried out using chemicals such as trichloroacetic acid and phenol. An inability to control the depth of the peeling, possible pigmentary change, and risk of scarring are among the problems associated with chemical peeling.

All the above methods suffer from the problem of being invasive and involve significant amount of pain. As these cosmetic procedures are all generally elective procedures, pain and the occasional side effects have been a significant deterrent to many, who would otherwise like to undergo these procedures.

To overcome some of the issues associated with the invasive procedures, laser and radio frequency energy based wrinkle reduction treatments have been proposed. For example, U.S. Pat. No. 6,387,089 describes using pulsed light for heating and shrinking the collagen and thereby restoring the elasticity of the skin. Since collagen is located within the dermis and subcutaneous layers and not in the epidermis, lasers that target collagen must penetrate through the epidermis and through the dermal epidermal junction. Due to Bier's Law absorption, the laser beam is typically the most intense at the surface of the skin This results in unacceptable heating of the upper layers of the skin. Various approaches have been described to cool the upper layers of the skin while maintaining the layers underneath at the desired temperature. One approach is to spray a cryogen on the surface so that the surface remains cools while the underlying layers (and hence collagen) are heated. Such an approach is described in U.S. Pat. No. 6,514,244. Another approach described in U.S. Pat. No. 6,387,089 is the use of a cooled transparent substance, such as ice, gel or crystal that is in contact with the surface the skin. The transparent nature of the coolant would allow the laser beam to penetrate the different skin layers.

To overcome some of the problems associated with the undesired heating of the upper layers of the skin (epidermal and dermal), U.S. Pat. No. 6,311,090 describes using RF energy and an arrangement comprising RF electrodes that rest on the surface of the skin. A reverse thermal gradient is created that apparently does not substantially affect melanocytes and other epithelial cells. However, even such non-invasive methods have the significant limitation that energy cannot be effectively focused in a specific region of interest, say, the dermis.

Other approaches have been described to heat the dermis without heating more superficial layers. These involve using electrically conductive needles that penetrate the surface of the skin into the tissue and provide heating. U.S. Pat. Nos. 6,277,116 and 6,920,883 describe such systems. Unfortunately, such an approach results in widespread heating of the subcutaneous layer and potentially melting the fat in the subcutaneous layer. This leads to undesired scarring of the tissue.

One approach that has been described to limit the general, uniform heating of the tissue is fractional treatment of the tissue, as described in published U.S. Patent Application 20050049582. This application describes the use of laser energy to create treatment zones of desired shapes in the skin, where untreated, healthy tissue lies between the regions of treated tissue. This enables the untreated tissue to participate in the healing and recovery process.

Hence, it will be desirable to accomplish the fractional or patterned heat generation in the epidermis, dermis or subcutaneous layers of the skin using needles or microneedles that could be located at the desired depth in the skin.

### SUMMARY OF THE INVENTION

The invention describes improved methods and systems for rejuvenating aging skin to achieve cosmetically desirable outcomes by shrinking collagen using radio frequency energy that is delivered to the target sites using a microneedle electrode array.

The invention provides a dermatological treatment apparatus for selectively treating zones of tissue within the skin. Such selective tissue treatment is achieved using an array of electrically conductive microneedles that are connected to a radio frequency energy source. The RF energy source is operated by a controller unit, which is programmable and is capable of activating a selected group of needle electrodes. This programmable selectivity leads to a desired pattern of microneedle electrodes treating zones of tissue at the desired location in the skin and simultaneously sparing tissue that is surrounding the targeted zones.

The controller unit has the capability of monitoring changes in the tissue parameters, such as conductivity and temperature, and uses these measurements to determine when treatment should be terminated. Additionally, the tissue property measurements can identify sensitive zones, such as nerves, to be excluded from the thermal treatment.

The microneedles can also be hollow and thereby are capable of delivering desirable therapeutic agents to the treated zones. The therapeutic agents could include anesthetics, growth factors, stem cells, botulinum toxin, etc.

In another embodiment, the microneedles are driven into the tissue using mechanical energy, where such driving force could be vibration or pressure. In another aspect of this invention, the treatment device has a suction coupling such that the each microneedle penetration depth could be individually controlled. This is highly desirable in anatomical regions containing uneven contours, such as the face and the transition areas from the face to the neck.

In yet another embodiment of this invention, the controller has algorithms embedded in it, which identifies the appropriate needle pair(s) that needs to be activated so that there is enough thermal relaxation time at the treated zones and thereby avoiding overheating of the treated zones and maintaining the desired temperature of the untreated tissue surrounding the treated zones.

Additional features and advantages of the invention described in the drawings and the description below and in the appended claims.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 is a diagram showing an embodiment of the invention wherein a handpiece is placed in contact with the skin. Vacuum channels are used to make reproducible contact with the skin surface and to force the needles into the skin. RF energy is delivered to the skin through the needles to form fractional treatment zones. Cryogenic spray is used to cool the needles and/or the contact plate to prevent overheating of selected tissue.

FIG. 2 is a diagram showing details of the vacuum channel in the area around the needle array.

FIGS. 3A and 3B show wiring diagrams of the needle electrode array for two embodiments of the invention. FIG. 3A shows a wiring pattern where only two source electrodes are used. FIG. 3B shows a wiring pattern where multiple source electrodes are used such that each electrode is wired individually.

FIGS. 4A, 4B and 5A, 5B are treatment patterns that can be created using either of the wiring patterns shown in FIGS. 3A and 3B. FIGS. 4A and 5A show treatment patterns and the electrodes. FIGS. 4B and 5B show the corresponding treatment patterns after the electrodes have been removed from the skin The treatment pattern in FIG. 4B is discontinuous. The treatment pattern in FIG. 5B is continuous.

FIGS. 6A, 6B and 7A, 7B show treatment patterns that can be created using the wiring pattern shown in FIG. 3B. FIGS. 6A and 7A show treatment patterns and the electrodes. FIGS. 6B and 7B show the corresponding treatment patterns after the electrodes have been removed from the skin.

FIGS. 8A and 8B show a treatment pattern that is used to treat an unwanted blood vessel.

FIGS. 9A and 9B show a treatment pattern that can be created using either of the wiring patterns shown in FIGS. 3A and 3B, if the device is elongated in one direction of the array relative to the other. FIG. 9A shows a treatment pattern and the electrodes. FIG. 9B shows the corresponding treatment pattern after the electrodes have been removed from the skin.

FIG. 10 is a diagram of the lines of maximum extensibility for the face. Treatment can be performed along the lines of maximum extensibility to enhance the treatment appearance.

FIG. 11 is a diagram of an embodiment of the invention wherein the micro needles have shallow penetration.

FIG. 12 is a diagram of an embodiment of the invention wherein the micro needles are hollow to allow delivery of a substance into the skin tissue.

FIG. 13 is a diagram of an embodiment of the invention wherein the depth of the needles can be adjusted by adjusting the space between two plates. In this embodiment, the needles may be pushed into the skin with the assistance of vacuum.

FIGS. 14A and 14B show an embodiment of the invention that comprises a removable tip that attaches to a handpiece.

FIGS. 15A and 15B show histology sections of human skin stained with hemotoxylin and eosin following ex vivo treatment with RF energy delivered using a microneedle electrode array. FIG. 15A and 15B represent different pulse conditions for the pulse source and the needle positions.

### DETAILED DESCRIPTION OF THE PREFERRED EMBODIMENTS

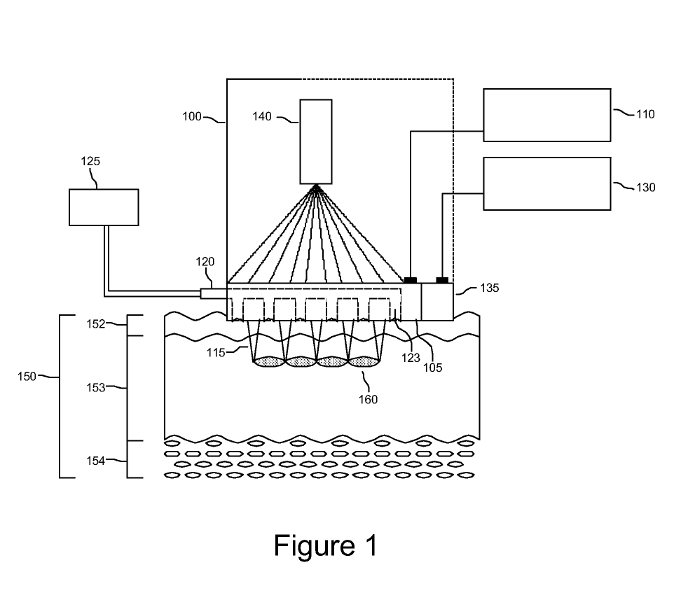

FIG. 1 illustrates an embodiment of the invention. In this embodiment, a radio frequency (RF) source 110 is connected to an array of needles 115 that are mounted on a contact plate 105. The RF source 110 generates energy that is delivered to the tissue to create treatment zones 160 within the skin 150. A vacuum apparatus or suction apparatus 125 is attached to a vacuum port 120 of the contact plate 105. A handpiece 100 is used by the practitioner to control the location of the device on the skin and deliver RF energy to the desired location for treatment. A cryogenic spray 140 is used to cool the contact plate 105 and/or the needles 115. A vibrating element 135 is mechanically coupled to the contact plate 105. A vibration power source 130 is preferably located external to the handpiece 100 and is connected to the vibrating element 135 to power the vibrating element 135, which in turn would drive the needles 115 into the skin, as the vibrating element 135 is mechanically coupled to the contact plate 105 on which the needles are mounted.

The handpiece 100 can be applied to the surface of the skin 150. This causes the needles 115 to penetrate the surface of the skin 150. The skin 150 may have significant wrinkles or other topology. Therefore, the needles may not all penetrate to the same depth within the skin. In some embodiments of the invention, it is preferable that all of the needles penetrate to a predetermined depth within the skin 150. Preferably, the needles are arranged to primarily deliver treatment in the papillary dermis and/or the upper reticular dermis. A vacuum port 120 can be attached to a vacuum apparatus 125. The vacuum apparatus 125 creates a negative pressure within the vacuum channels 123 (as shown in FIG. 1) so that the surface of the skin 150 is drawn into contact with the contact plate 105 and the needles 115 penetrate into the skin 150 to a predetermined depth beneath the surface of the skin 150. The vibrating element 135 can be powered by the vibration power source 130 to help the needles 115 penetrate into the skin 150 more easily by vibrating the contact plate 105 and/or the needles 115. While the needles 115 are located within the skin 150, they can be powered by the RF source 110 to create an array of treatment zones 160 through resistive heating of the tissue. In some embodiments, it may be desirable to avoid or limit treatment of the epidermis 152 or selected upper layers of the skin 150, which can be accomplished by cooling the back of the contact plate 105 with a cryogenic spray 140. It may also be desirable to limit the penetration depth of the needles such that the needles do not penetrate into the layer of subcutaneous fat 154 because melting of the fat layer can lead to scarring due to fat atrophy. Melting of the subcutaneous fat 154 also reduces the skin thickness which may not be desirable. The contact plate 105 can be thermally conductive to carry the heat away from the skin 150 during or following treatment.

Cryogenic spray cooling 140 may be used to actively cool the contact plate 105 to enhance the cooling of the skin 150. A cryogenic spray cooling 140 may also be used to cool the surface of the skin 150 directly by spraying cryogen onto the surface of the skin 150. The cryogenic spray 140 could be a container containing a cryogen, such as, for example, compressed tetrafluoroethane.

In an alternate embodiment (not shown), the contact plate 105 may be cooled by circulating a liquid at room-temperature or chilled liquid to make thermal contact to the contact plate 105. The cooling fluid can conductively cool the needles 115 and/or the contact plate 105, which lowers the temperature of the skin 150 relative to the temperature that would be achieved without cooling. Cooling of the skin 150, when desired, can thus be used to avoid heating or over heating of the epidermis 152 or upper layers of the skin 150.

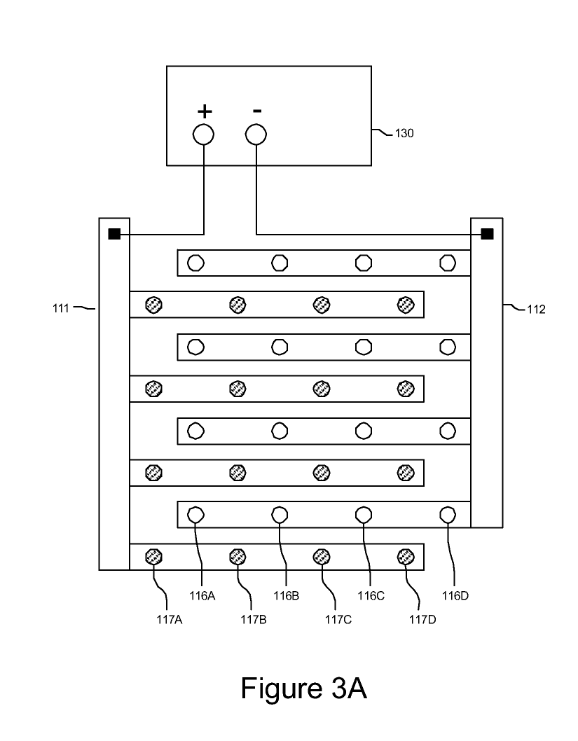

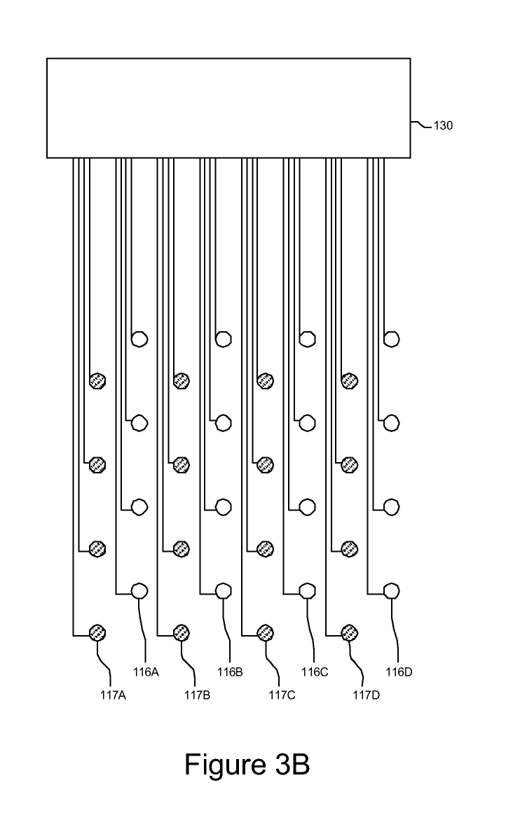

In a preferred embodiment, the needles 115 are 36-gauge electrically conductive needles that are connected to the RF source 110. The needles 115 could be prepared by cutting commercially available long hypodermic needles . The needles 115 can be soldered onto a circuit board where the circuit board is patterned, as shown in the patterns of FIG. 3, to create an array of wired needles 115 that can be used according to this invention. The circuit board can be single or multilayered.

Preferably, the needles 115 are pointed and made of a solid conductive material such as, for example, metal. The needles 115 may also be hollow or made of an electrically nonconductive material that has a conductive coating. In some embodiments, each of the needles 115 can comprise an electrically conductive shaft or coating that is coated on the surface with an electrically non-conductive material such as, for example, Teflon. An electrically non-conductive coating material can be patterned in order to channel the RF treatment energy to a particular location where the electrically conductive shaft or coating contacts the skin through a gap in the patterned electrically non-conductive coating. In a preferred embodiment, the needles 115 are 50 to 300 μm in diameter. The diameter of the needles 115 is preferably at least 50 μm to reduce breakage of the needles 115. The diameter of the needles 115 is preferably less than 300 μm to allow close packing of the needles 115 and to reduce disruption to the skin 150 and purpura, as the needles 115 penetrate into the skin 150. The needles are also described as microneedles and when connected to an RF energy source as a microneedle electrode array.

The RF source 110 can be a radio frequency or microwave source that is used to create a temperature increase in the tissue when used with the needles 115. The RF source 110 may be bipolar or monopolar. Preferably, these sources operate in a frequency range used for industrial applications so that cheaper electromagnetic sources are available. For example, the frequency of the RF source can be chosen to be about 6.78 MHz or about 13.56 MHz. In some preferred embodiments, the frequency range is from 0.1 to 10 MHz or from 0.4 to 3 MHz. The resistance of the skin varies with the frequency of RF source. The frequency range of the RF source can be chosen based on the desired treatment zone profile including for example treatment zone size, treatment zone shape, treatment zone aspect ratio, and treatment zone spacing.

In a preferred embodiment, the vacuum channels 123 are machined into the contact plate 105. The contact plate 105 is preferably electrically-insulating to prevent shorting between the needles while providing physical support for the needles. An electrically insulating material that could be used in some embodiments is alumina. A vacuum port 120 connects to the vacuum channels 123 to create a negative pressure in the vacuum channels 123 when the vacuum port 120 is connected to the vacuum apparatus 125. In a preferred embodiment, the vacuum port 120 is a hose fitting to which a vacuum hose is attached to connect the vacuum channels 123 to the vacuum apparatus 125. The vacuum apparatus 125 can be, for example, a vacuum pump.

In a preferred embodiment, the vibrating element 135 is a piezo-electric vibrating unit or an electrical buzzer and the vibration power source 130 is an electrical source that is matched to the vibrating element 135.

The treatment zones 160 are shown in FIG. 1 to be located within the dermis 153, but these treatment zones may also be located within the epidermis 152, at the dermal-epidermal junction, or treatment zones may include regions in both the epidermis 152 and dermis 153.

The treatment pattern created by the treatment zones 160 can depend, for example, on the distribution of the needles 115, on the wiring patterns for the needles 115, and/or on the firing pattern of the needles 115 by the RF source 110. The array of treatment zones 160 that is created according to the invention may be regular or irregular. It will typically be easier to design and build an apparatus using automated manufacturing techniques if the array of treatment zones 160 is regular. Creating irregular arrays of treatment zones 160 will reduce the visual impact due to treatment by making the treatment appear more natural since many natural features vary in an irregular manner within the skin 150.

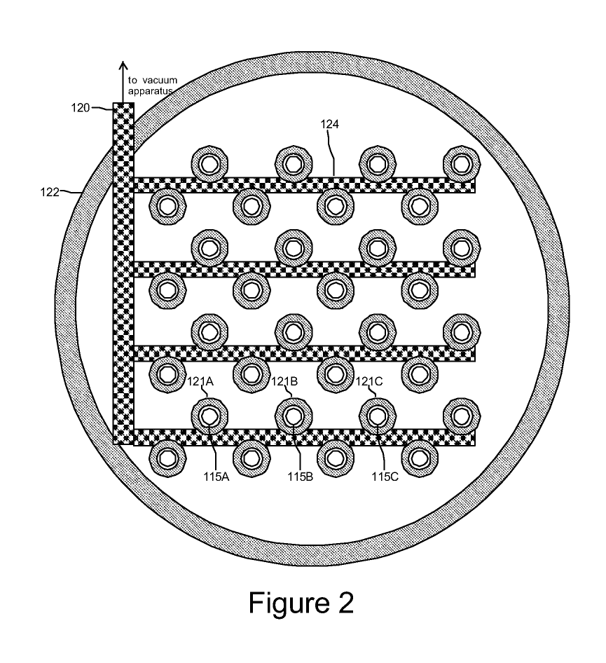

The vacuum channels 123 shown in FIG. 1 can be arranged according to the desired treatment application. A preferred embodiment for the geometry of the vacuum channels 123 of FIG. 1 is shown in FIG. 2. In this embodiment, the vacuum channels 123 comprise an outer vacuum ring 122, vacuum feeder lines 124, and individual needle-specific vacuum rings 121. In FIG. 2, negative pressure created within the outer vacuum ring 122 holds the skin to the contact plate 105 shown in FIG. 1 to hold the skin 150 and contact plate 105 in contact as shown in FIG. 1. The outer vacuum ring 122 creates a more uniform application of force by the individual needle-specific vacuum rings 121. In this embodiment, the vacuum feeder lines 124 are not typically in contact with the skin 150, but they can be. The vacuum feeder lines 124 are used to connect the vacuum port 120 to the outer vacuum ring 122 and to the individual needle-specific vacuum rings 121.

Individual needle-specific vacuum rings 121A-C wrap around each of the needles 115A-C in the array. The negative pressure created within each of the needle-specific vacuum rings 121 forces the skin 150 onto the encircled needle such that the encircled needle penetrates to a predetermined depth in the skin 150.

The RF source 110 shown in FIG. 1 may be wired to the needles 115 in different patterns that may be chosen based on the desired application. A preferred embodiment of the wiring pattern is shown in FIG. 3A. In FIG. 3A, the RF source 110 has two output terminals. One of the output terminals is labeled with a plus sign (active) and the other with a minus sign (return) to indicate two poles of the RF source 110. Alternate interleaved rows of the array are wired to either the plus or the minus electrode through the common wiring buses 111 and 112. Thus, two interleaved arrays of regularly spaced needles 115 are formed. One array includes all of the negative polarity needles 116 (return electrodes) and the other includes all of the positive polarity needles 117 (active electrodes). In FIG. 3, the negative needles 116 are open and the positive needles 117 are shaded.

The spacing between the negative needles 116 and the positive needles 117 can be chosen, for example, based on the resistance of the skin at the frequency of the RF source 110 such that the pulsing of the RF source 110 creates a treatment zone 160 between nearest neighbors within the array of needles 115.

Note that needles 115 can be described generally as needles 115 or they can be further categorized as positive polarity needles 117 (shaded in FIGS. 3-9) and negative polarity needles 116 (unshaded in FIGS. 3-9). Positive needles 117 and negative needles 116 are subsets of the general category of needles 115. Positive and negative polarities refer to opposite poles of the RF source.

In an embodiment, the array of needles 115 comprises at least sixteen needles 115. The use of at least sixteen needles makes the treatment proceed faster than with fewer needles and also helps to reduce the torque that may be applied to each needle which could tear the skin 150.

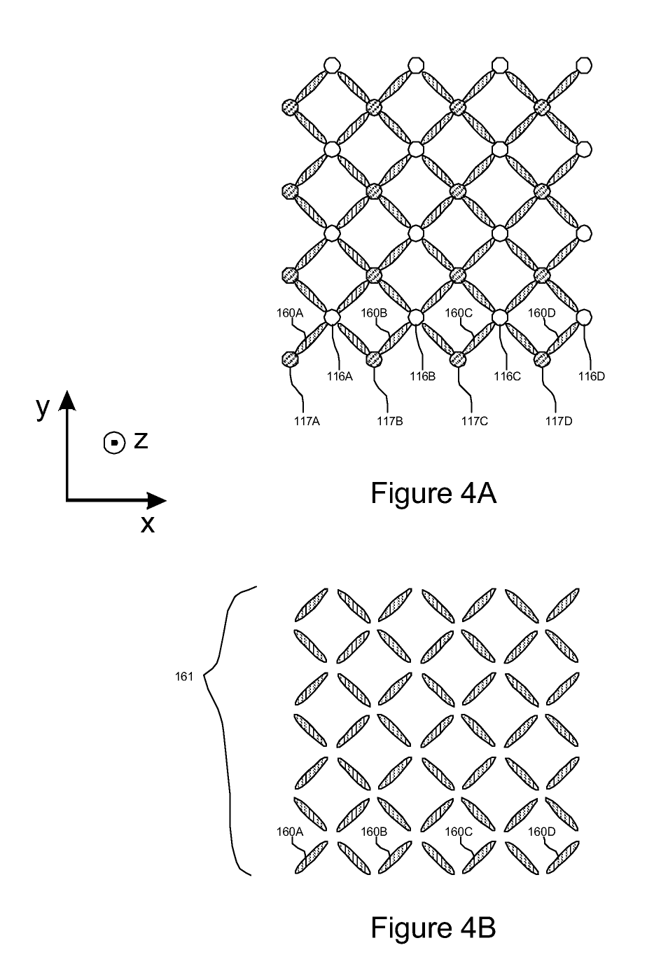

FIGS. 4A and 4B show a treatment pattern 161 of treatment zones 160 that can be produced from either of the wiring patterns shown in FIG. 3A or 3B. FIG. 4A shows the treatment zones 160 that are created between nearest neighbor needles 115 that are connected to opposite poles of the RF source 110. FIG. 4B shows the corresponding treatment pattern 161 of FIG. 4A after the needles have been removed from the skin 150. The treatment pattern 161 is an example of a discontinuous treatment pattern 161 of treatment zones 160.

With the proper choice of parameters, the treatment can be self limiting to create treatment zones 160 of approximately uniform size across the treatment pattern 161. The self limiting nature of the treatment can be achieved by choosing the frequency of the RF source 110 to be a frequency for which the tissue resistivity (impedance) increases as the tissue is treated. As skin 150 is treated, the water content of the treatment zone 160 is reduced, which typically increases the resistivity of the treatment zone 160 relative to the surrounding skin 150.

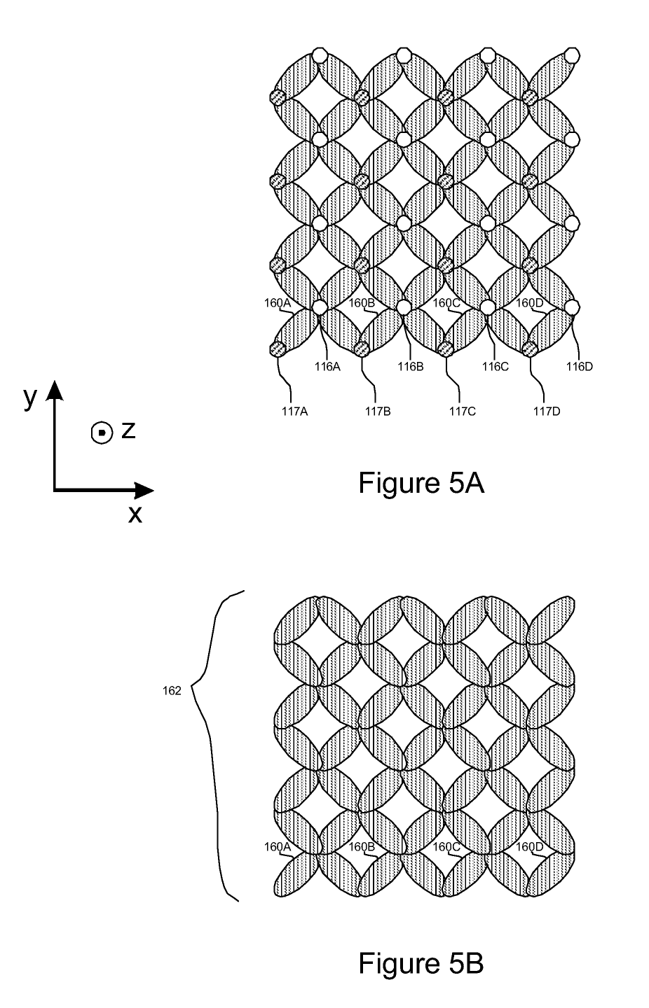

At high RF pulse energies and/or close spacing of the array of needles 115, the treatment zones 160 can be created such that the treatment zones 160 merge together to form a continuous treatment pattern 162 as shown in FIGS. 5A and 5B. The continuous treatment pattern 162 can be created using either the wiring pattern shown in FIG. 3A or FIG. 3B.

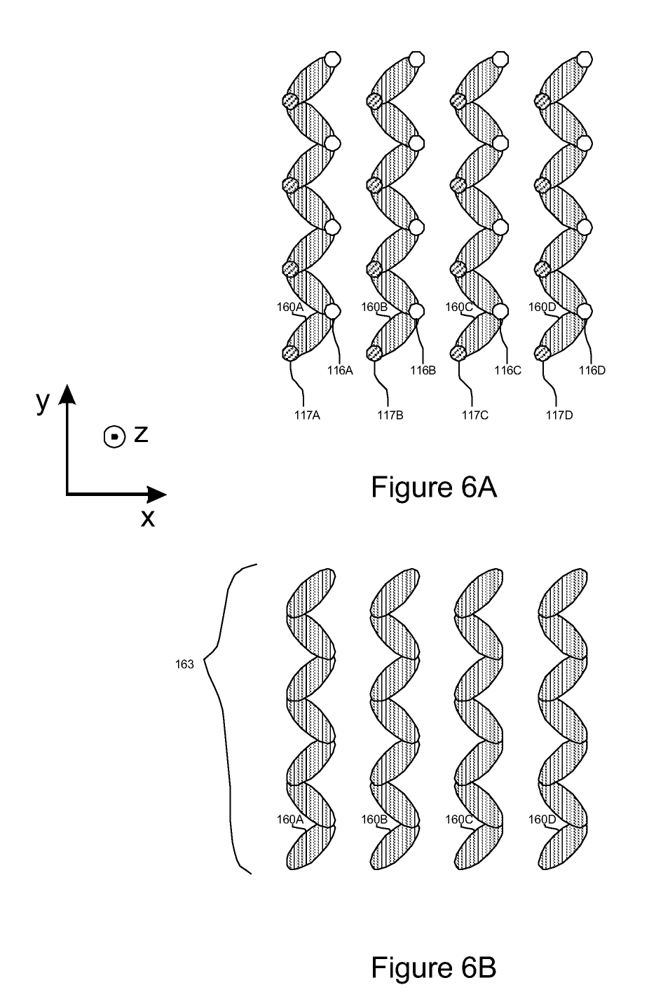

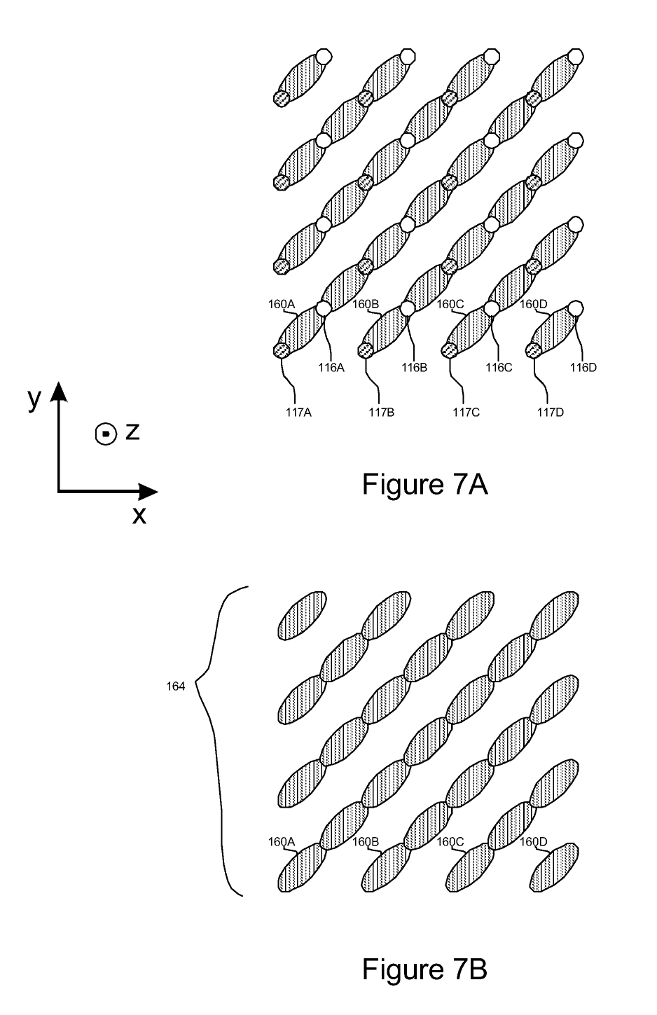

FIGS. 6A, 6B and 7A, 7B show other treatment patterns 163 and 164 that can be created using the wiring pattern shown in FIG. 3B. In these embodiments, not all of the electrode pairs are activated. The treatment patterns 163 and 164 differ in the timing between pulsing of electrode pairs to create each treatment zone 160 and in which electrode pairs are pulsed.

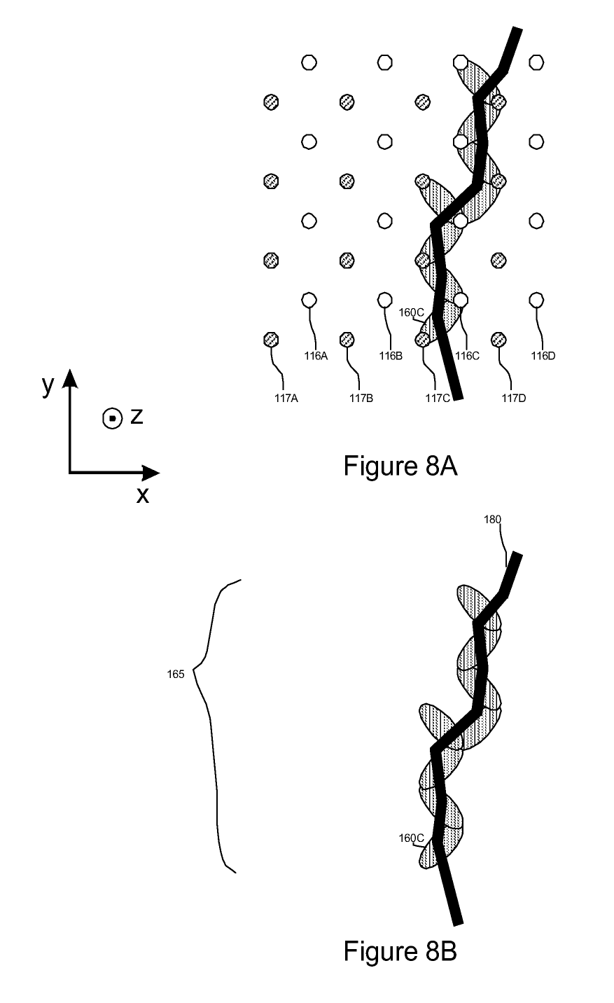

In an alternate embodiment, the needles are connected to an RF switching network such that the polarity of each needle 115 can be selected for each pulse of the RF source 110. Selected needles 115 may also be floated or grounded by the switching network to create other treatment patterns. The array of needles 115 can thus be reconfigurable. A reconfigurable array of needles 115 can be used to actively target features within tissue. For example, a CCD camera or visual observation port can be used to identify the position of a blood vessel 180 to be treated within the skin 150. As shown in FIGS. 8A and 8B, once the blood vessel 180 has been identified, selected needle pairs can be fired to treat or to spare the identified blood vessel 180. Other identifiable objects within or on the skin 150 can be targeted or spared using a reconfigurable array of needles 115. For example, sebaceous glands, tattoos, wrinkles, scars, hairs, hair follicles, and pigmented lesions may be targeted using reconfigurable arrays of needles 115. Another example of a reconfigurable array of needles 115 is an individually addressable needle system as shown in FIG. 3B where the RF source 110 can individually address each needle or selected sets of needles within in the array. Apart from visual identification, structures such as blood vessels could also be identified by commonly known techniques. One such technique would be an impedance sweep of the tissue.

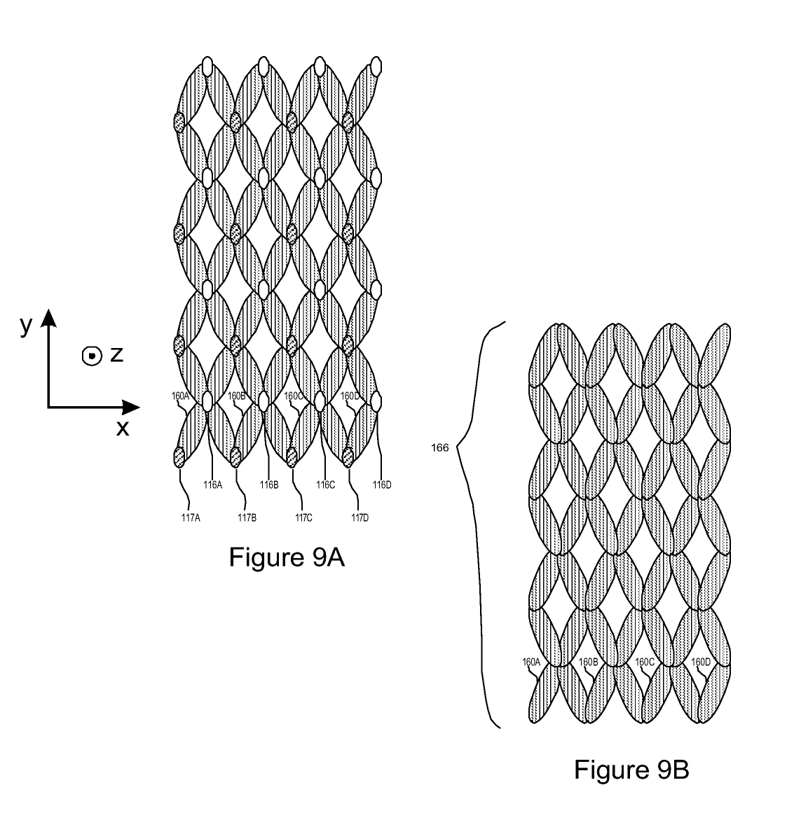

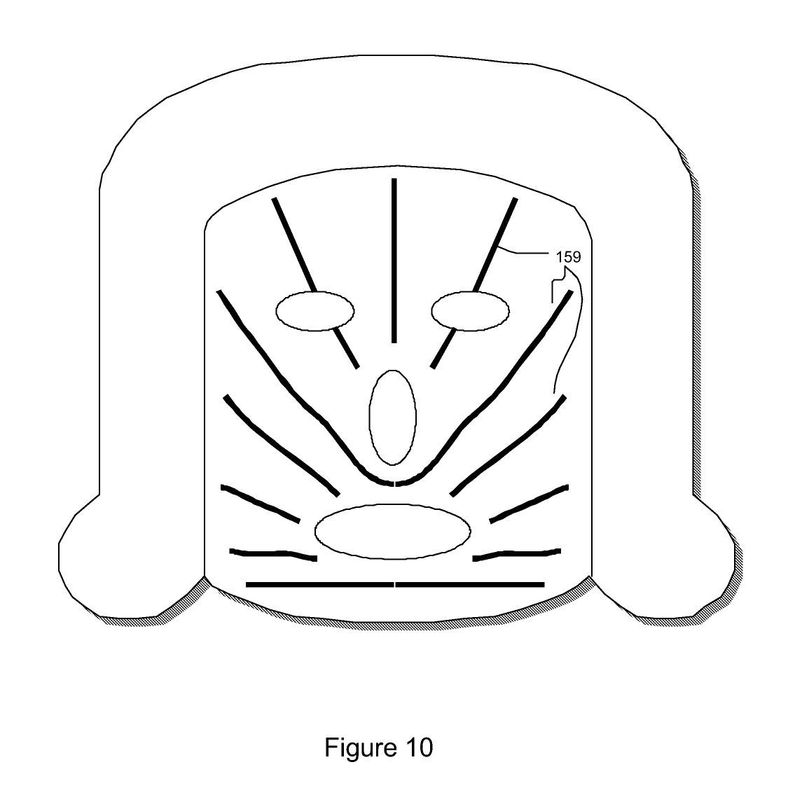

FIG. 9 shows an arrangement of the needles 115 in which the treatment pattern 165 is elongated due to a different arrangement of the needles 115. Also illustrated in this example is the use of needles 115 with oval cross sections, which can be used to create more localized electrical field profiles within the tissue or to create a discontinuous treatment pattern 161 as shown in FIG. 4B. Oval cross sections can also be used to reduce local fields and thus reduce charring and over-treatment.

Each treatment zone 160 can be created by electrically connecting the needles 116 and 117 at the opposite ends of each local region of skin 150 to be treated to different poles of the RF source 110. One or more treatment zones 160 within any of the treatment patterns 161-166 can be created either sequentially or simultaneously depending on the desired application. Sequential creation of treatment zones 160 is useful in situations where minimizing thermal crosstalk is important or where the power of the RF source 110 is limited. Simultaneous creation of treatment zones 160 is useful in situations where treatment speed is important.

Each of the treatment patterns 161-166 desirably spares healthy tissue between the treatment zones 160. Sparing of healthy tissue between treatment zones 160 reduces the incidence of scarring and promotes rapid healing by allowing nutrients, cells, and cytokines to flow more quickly to the wounded areas to stimulate the wound healing response. The spared tissue also allows transport to the dermal-epidermal junction and the epidermis so that the epidermis can remain healthy or heal quickly following treatment.

The treatment patterns 161-166 are shown here as examples of treatments that can be created performed according to the invention. Other patterns can be used to create different effects based on particular applications.

The treatment pattern 164 shown in FIGS. 7A and 7B is particularly useful because it can create a line of tension within the skin 150 due to collagen denaturation. Collagen denaturation causes collagen fibers to shrink in length by up to approximately 60% or 70% and thus can provide considerable tension along a particular direction. To enhance the appearance of shrinkage on the skin, the treatment can preferably be aligned to cause shrinkage along the directions of maximum extensibility. The lines of maximum extensibility 159 are illustrated in FIG. 10. Arranging treatment along the lines of maximum extensibility 159 will be helpful for reducing the visibility of wrinkles

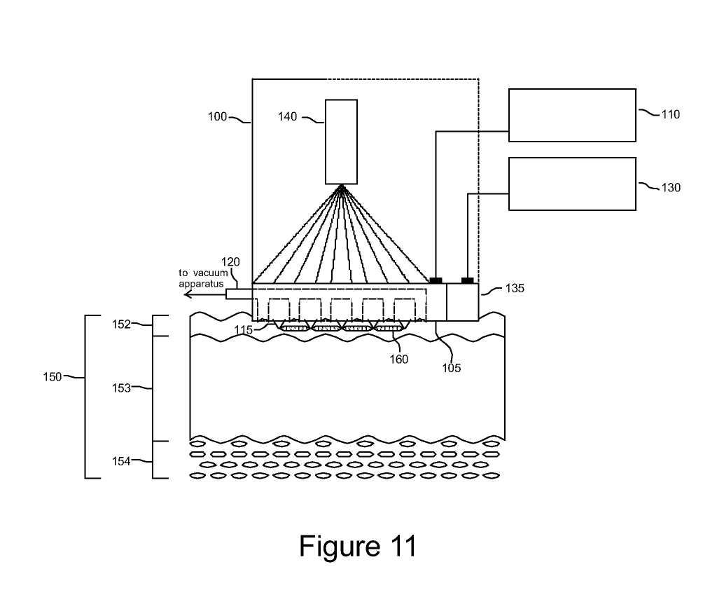

FIG. 11 shows an embodiment of the invention in which needles 115 penetrate primarily to predetermined depths within the epidermis 152 such that treatment zones 160 are created in the epidermis 152 and/or along the dermal-epidermal junction located at the base of the epidermis 152. To limit the penetration to only the epidermis, it may be desirable to limit the predetermined needle penetration depth to 5-50 μm.

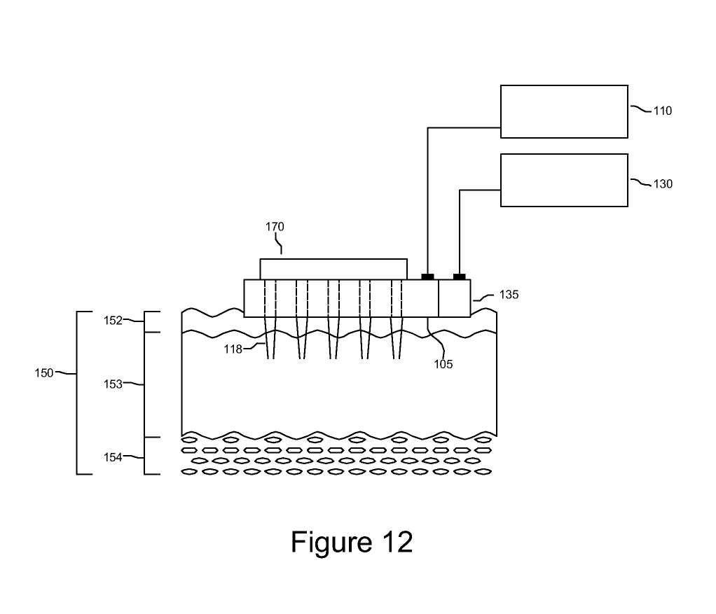

FIG. 12 shows an embodiment of the invention in which delivery needles 118 are hollow and open at the distal end. Delivery needles 118 can be physically connected to a fluid filled reservoir 170 that contains a therapeutic substance that is to be delivered beneath the surface of the skin into, for example, the epidermis 152, dermis 153, subcutaneous fat 154, or muscular layers (not shown). Examples of therapeutic substances that can be delivered are anesthetics (such as lidocaine), vitamins (such as vitamin C), minerals, growth factors, pro-drugs, hormones, stem cells, vasoconstrictors, steroids, botulinum toxin, and photosensitive toxins. In an alternate embodiment, the needles can be made to be permeable so that therapeutic substances can be delivered through the permeable needles.

Since the primary barrier for many topically applied therapeutic substances is the stratum corneum, which is the outermost layer of the epidermis, the delivery needles 118 can significantly enhance delivery of a therapeutic substance even if the delivery needles 118 only penetrate into the epidermis 152 and not into the dermis 153.

The delivery of botulinum toxin in combination with the RF treatment using a microneedle area is one embodiment, whereby the combination treatment of fractional RF tightening of tissue and local temporary paralysis of the underlying muscles through the use of botulinum toxin is effective for treatment of wrinkles and the delay of recurrence of wrinkles.

In an alternate embodiment, therapeutic substances can be applied to the surface of the skin 150 after treatment to cause the therapeutic substances to penetrate into the pores or channels created by needles 115 or 118.

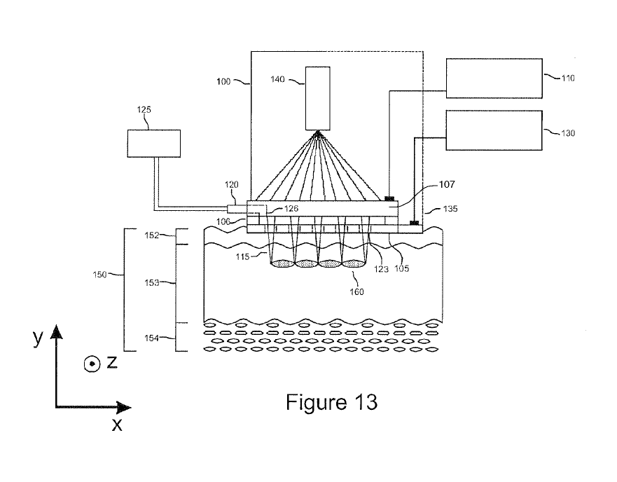

In some embodiments, it may be desirable to use a high level of treatment to create large treatment zones or allow a large needle separation. In such embodiments, the skin may be charred or over-treated due to the local concentration of the electric field that occurs, for example, near the ends of the needles where the electric field may be highest. As shown in FIG. 13, the incidence of over-treatment or charring can be reduced by cooling the needles 115 using the cryogenic spray 140 by spraying directly onto a thermal mounting plate 107 that is thermally connected to the needles 115. The embodiments that use this cooled needle approach can also reduce the occurrence of the skin 150 adhering to the surface of the needles 115 when the RF treatment is performed. The contact plate 105 may be thermally insulating or thermally conductive depending on the desired thermal profile for treatment. Chilling the needles 115 will help to reduce purpura in some applications.

The contact plate 105 may be in thermal contact with the thermal mounting plate 107 to cool the surface of the skin 150 instead of or in addition to cooling the needles 115. In another embodiment, the cryogenic spray 140 may also be directed to cool both the contact plate 105 and the thermal mounting plate 107 by patterning a first plate, which is either the contact plate or the thermal mounting plate 107, such that part of the cryogen emanating from the cryogen spray 140 passes through patterned regions in the first plate to cool the second plate that lies beyond the first plate.

In some embodiments, it may be desirable to use vacuum force to push the needles 115 into the skin 150 after good contact has been established between the contact plate 105 and the skin 150. The embodiment shown in FIG. 1 is a preferred embodiment, as it does not have many moving parts that can wear out. An alternate embodiment shown in FIG. 13 provides better contact between the contact plate 105 and the skin 150 prior to the activation of the vacuum apparatus 125.

In FIG. 13, the vacuum apparatus 125 draws a negative pressure to create a force between the thermal mounting plate 107 and the contact plate 105. The vacuum apparatus 125 is connected to the chamber between the thermal mounting plate 107 and the contact plate 105 via the vacuum port 120 and the vacuum feeder line 126. The needles 115 can be attached to the thermal mounting plate 107. As the chamber is pumped to a negative pressure, the force between the thermal mounting plate 107 and the contact plate 105 can be used to force needles 115 to a predetermined depth within the skin 150. The adjustable spacer 106 may comprise bellows that can be expanded or compressed to create the desired offset to control the penetration depth of the needles. By adjusting the height of the adjustable spacer 106, the predetermined depth of penetration of the needles 115 in the skin 150 can be adjusted.

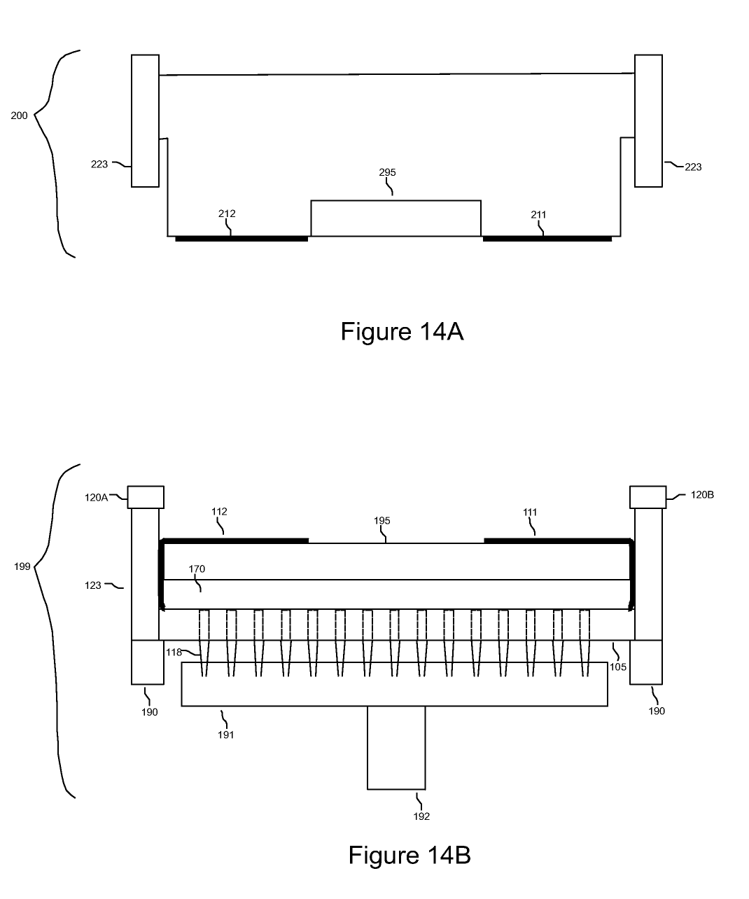

FIGS. 14A and 14B show an embodiment of the invention that contains a disposable tip 199. Delivery needles 118 are attached to a contact plate 105 for delivery of a therapeutic substance from the fluid filled reservoir 170. Vacuum channels 123 are connected to two vacuum ports 120A, and 120B for connection to handpiece 200 that contains or attaches to a vacuum apparatus (not shown). The disposable tip 199 also comprises two electrical contact pads 111 and 112 for making electrical contact to two corresponding electrical contact pads 211 and 212 that are located on the handpiece 200. The electrical contact pads 211 and 212 are connected to an RF source (not shown). The other end of the electrical contact pads 111 and 112 are connected to the delivery needles 118. The tip 199 can be attached to a handpiece 200 using a magnetic latch 195 or by snap fitting or by other mechanical means. The needles 118 are surrounded by a vacuum curtain 190 that makes a vacuum seal with the skin (not shown) during treatment. Prior to use, the delivery needles 118 can be protected using a protective needle plug 191 that includes a plug handle 192 for removing the needle plug 191 from the delivery needles 118.

To use the tip 199 shown in FIG. 14B for treatment, the tip 199 is attached to the handpiece 200 using the magnetic latch 195. The contact pads 111 and 112 make electrical contact to the corresponding electrical contact pads 211 and 212 on the handpiece 200. The vacuum channels 223 attach to the vacuum ports 120 on the tip 199. The protective needle plug 191 is removed using the plug handle 192. The delivery needles 118 of the tip 199 are then applied to the skin (not shown) using manual pressure on the handpiece 200. The vacuum curtain 190 would make an air tight seal with the skin. To help make the seal air tight, a vacuum compatible gel, grease, or sealant can be used. The vacuum apparatus (not shown) is activated to create a negative pressure between the contact plate 105 and the skin (not shown) to force the delivery needles 118 into the skin to a predetermined depth. The RF source (not shown) is then pulsed to create treatment zones (not shown) within the skin. Following treatment, the handpiece 200 is lifted from the skin to withdraw the delivery needles 118 and remove the tip 199 from the skin. The tip 199 can then be manually detached from the handpiece 200.

The vacuum curtain 190 can be made of vinyl and should be thin enough to flex without breaking when applied to the skin so that a good vacuum seal can be created.

A fast-acting anesthetic in conductive saline solution can be added to the fluid-filled reservoir 170 for management of pain during or after the RF treatment. The use of conductive saline solution enlarges the electrical path for the RF treatment.

The tip 199 can be sterilized, if materials are chosen that are compatible with sterilizers, such as stainless steel and high melting temperature plastics.

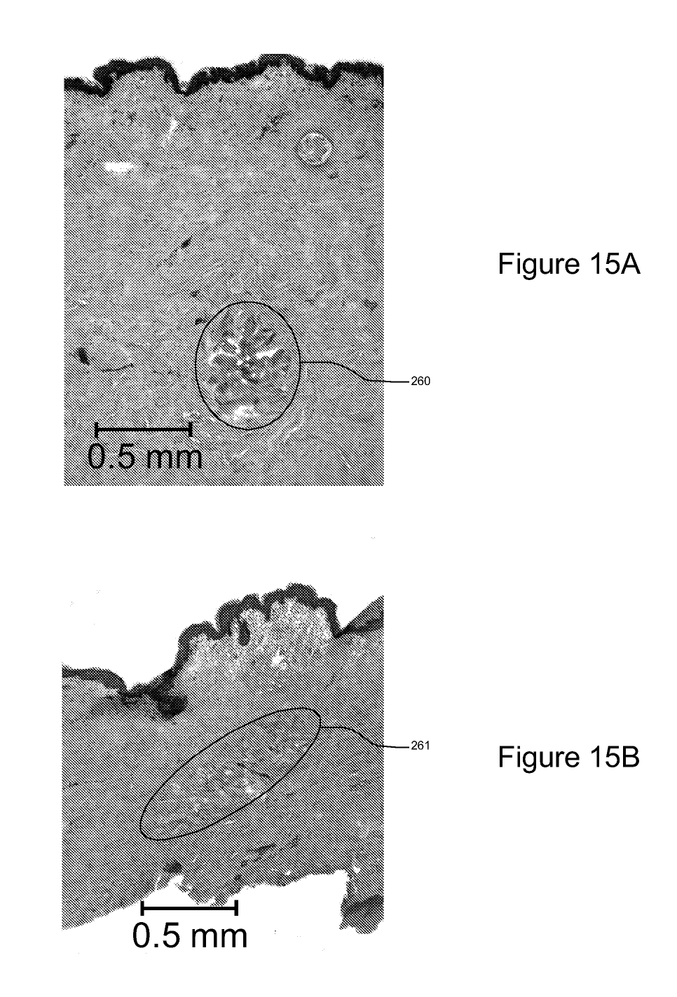

FIG. 15 shows several treatment zones 260, 261 created using an ex vivo human tissue model. Excised human abdominal skin 150 was placed on a hot plate to heat the skin 150 to approximately body temperature prior to treating using an RF source 110 connected to a pair of needle probes 115. Saline soaked gauze sheets were used to keep the skin tissue moist as it was being heated prior to treatment. Two needles 115 were used to demonstrate the treatment zones created by each needle pair 116 and 117.

Ex vivo tissue samples were frozen in optimal cutting temperature fluid (International Medical Equipment, Inc., San Marcos, Calif.) and were sliced with a cryostat into approximately 6-15 μm thick sections and stained with hematoxylin & eosin (Harris Hematoxylin and Eosin Y stains from International Medical Equipment, Inc.). The sliced sections were placed on glass microscope slides, dehydrated in 95% alcohol, and rehydrated in deionized water. Samples were then stained with hematoxylin to dye nuclei and cytoplasm within cells and with eosin to dye connective tissue. The concentration of alcohol was adjusted to optimize the contrast visible in the slide. Xylene was used to rinse the slides prior to mounting a glass coverslip.

FIG. 15A shows the results of using of a needle pair that penetrated approximately 1-2 mm into the skin with a needle separation of 0.5 mm A bipolar RF source operating at a frequency of 0.47 MHz, a power of 5 W, and a pulse duration of 400 ms was used. The treatment zone 260 that was created has dimensions of approximately 500 μm width and 600 μm height. The aspect ratio of width to height of the treatment zone 260 is therefore approximately 5:6.

For FIG. 15B, the conditions were similar to those for FIG. 15A except the pulse duration was 200 ms, the separation between the needles 115 was 1 mm, and the depth of needle penetration was approximately 0.5-1 mm The treatment zone 261 that was created has dimensions of approximately 900 μm width and 250 μm height. The aspect ratio of width to height for the treatment zone 261 is therefore approximately 3.6:1.

Other pulse parameters could be used. A preferred pulse source frequency is 0.47 MHz, but other frequencies can be used as described above. Other frequencies are particularly useful to create treatment zones of different shapes because the material resistivity of the skin is frequency dependent. Therefore, different frequencies will create different treatment zone shapes for otherwise equivalent pulse conditions. For each electrode pair that is fired to create treatment zones between the electrode pair, the pulse energy from the RF source 110 is preferably 0.1 to 8.0 J and more preferably in the range of 0.5 to 2.0 J. Pulse energies in the range of 0.02 to 0.10 J can be used in cases where needles are spaced close together. Preferably, the aspect ratio of width to height for the treatment zones 160 is in the range of 1:2 to 5:1 and more preferably in the range 2:1 to 4:1. Treatment zones 160 with an aspect ratio of width to height of greater than 1:1 are called “lateral treatment zones.” The height of the individual treatment zones 160 is preferably 0.1 to 0.5 mm The preferred width of the individual treatment zones 160 is 0.1 to 2.0 mm, and more preferably 0.5 to 1.0 mm The depth of the needle penetration into the skin 150 is preferably 0.025 to 2.0 mm and more preferably from 0.2 to 1.0 mm Preferably the needles 115 penetrate into the dermis or epidermis to directly heat dermal or epidermal tissue through resistive heating. Larger or smaller treatment zones are within the scope of the invention and the size and location of the treatment zones will be application specific. There are some applications, such as for example, tattoo removal or fat removal that treatment will extend down into the subcutaneous fat or deeper. The pulse conditions outlined here produce substantial lateral tightening of skin tissue and treat substantial portions of the dermal tissue. These parameters can be used to coagulate collagen within the skin and to kill or injure cells to stimulate the wound healing response in surrounding healthy tissue.

Although the detailed description contains many specifics, these should not be construed as limiting the scope of the invention but merely as illustrating different examples and aspects of the invention. It should be appreciated that the scope of the invention includes other embodiments not discussed in detail above. For example, the disposable tip embodiment can also be used with needles that do not deliver a therapeutic substance. Various other modifications, changes and variations which will be apparent to those skilled in the art may be made in the arrangement, operation and details of the method and apparatus of the present invention disclosed herein without departing from the spirit and scope of the invention as defined in the appended claims. Therefore, the scope of the invention should be determined by the appended claims and their legal equivalents. Furthermore, no element, component or method step is intended to be dedicated to the public regardless of whether the element, component or method step is explicitly recited in the claims.

In the claims, reference to an element in the singular is not intended to mean “one and only one” unless explicitly stated, but rather is meant to mean “one or more.” In addition, it is not necessary for a device or method to address every problem that is solvable by different embodiments of the invention in order to be encompassed by the claims.

What is claimed is:

1. A method of treating tissue in human skin, the method comprising:
causing a plurality of needles to penetrate the surface of the human skin and into the tissue beneath the surface of the human skin;
supplying radiofrequency energy from the needles to the tissue across a target area at a depth beneath the surface of the human skin; and
treating the tissue with the radiofrequency energy to form a pattern of treated tissue in treatment zones about the needles and simultaneously spare healthy tissue in untreated zones surrounding the treatment zones.

2. The method of claim 1 wherein the needles are arranged in a pattern so to form the pattern of the treatment zones and the untreated zones.

3. The method of claim 1 wherein the needles are wired with the radio frequency energy source so to form the pattern of the treatment zones and the untreated zones.

4. The method of claim 1 wherein the radio frequency energy source is configured to energize the needles with the radiofrequency energy so to form the pattern of the treatment zones and the untreated zones.

5. The method of claim 1 further comprising:
cooling the surface of the human skin to lower the temperature of the human skin when the tissue is treated with the radiofrequency energy.

6. The method of claim 5 wherein the cooling is used to avoid heating or over heating of the epidermis or upper layers of the human skin.

7. The method of claim 1 further comprising:
conductively cooling the needles to lower the temperature of the human skin when the tissue is treated with the radiofrequency energy.

8. The method of claim 1 wherein the needles are arranged in an array and are mounted on a contact plate, and further comprising:
drawing the surface of the human skin into contact with the contact plate with negative pressure applied to the surface of the human skin from vacuum channels in the contact plate.

9. The method of claim 8 wherein the drawing the surface of the human skin into contact with the contact plate promotes the penetration of the needles into the surface of the human skin and into the tissue beneath the surface of the human skin.

10. The method of claim 1 wherein causing the plurality of needles to penetrate the surface of the human skin and into the tissue beneath the surface of the human skin comprises:
vibrating the needles to promote penetration into the tissue.

11. The method of claim 1 wherein the pattern of the treatment zones and the untreated zones is located within the dermis of the human skin

12. The method of claim 1 further comprising:
predetermining the depth beneath the surface of the human skin to which the needles penetrate into the tissue.

13. The method of claim 1 further comprising:
delivering a therapeutic substance to the tissue treated with the radiofrequency energy.

14. The method of claim 1 wherein the therapeutic substance is delivered to the tissue from the needles.

15. The method of claim 1 further comprising:
terminating the treatment of the tissue with the radiofrequency energy upon sensing a predetermined endpoint.

16. The method of claim 1 further comprising:
monitoring changes in a tissue parameter for use in terminating the treatment of the tissue.

17. The method of claim 1 further comprising:
monitoring changes in a tissue parameter for use in identifying sensitive zones to be excluded from the treatment.

18. The method of claim 1 wherein supplying the radiofrequency energy from the needles to the tissue across the target area at the depth beneath the surface of the human skin further comprises:
energizing a first fraction of the needles with a positive polarity of the radiofrequency energy; and
energizing a second fraction of the needles with a negative polarity of the radiofrequency energy.

19. The method of claim 1 wherein the pattern comprises a fractional dermatological treatment, and an appearance of the human skin containing the tissue treated with the radiofrequency energy is rejuvenated.

20. The method of claim 1 wherein the pattern comprises a fractional dermatological treatment, the radiofrequency energy causing collagen to be denatured and causing shrinking of collagen fibers to result in tightened skin.

21. The method of claim 1 wherein the needles are arranged in a reconfigurable array for targeting and treating sebaceous glands, tattoos, wrinkles, scars, hairs, hair follicles, or pigmented lesions.

22. The method of claim 1 wherein the treatment zones are between 50 microns and 300 microns in diameter.

23. The method of claim 1 wherein a frequency of the radiofrequency energy is chosen to be one whereby a tissue resistivity increases as the tissue is treated.
